(19) United States
(12) Patent Application Publication
(10) Pub. No.: US 2024/0279367 A1
(43) Pub. Date: Aug. 22, 2024

(54) AMINO-MODIFIED CHIP, PREPARATION METHOD THEREFOR AND USE THEREOF

(71) Applicant: GeneMind Biosciences Co., Ltd., Shenzhen, Guangdong (CN)

(72) Inventors: Diewen FENG, Shenzhen (CN); Zhifeng LIN, Shenzhen (CN); Jicai FAN, Shenzhen (CN); Lei LIU, Shenzhen (CN); Fang CHEN, Shenzhen (CN); Qi WANG, Shenzhen (CN)

(21) Appl. No.: 18/020,915

(22) PCT Filed: Aug. 10, 2021
(86) PCT No.: PCT/CN2021/111672

§ 371 Date: Feb. 10, 2023

(30) Foreign Application Priority Data
Aug. 12, 2020 (CN) 202010807921.4

(51) Int. Cl. C08F 8/08; C12Q 1/6837

(52) U.S. Cl. CPC C08F 8/08; C12Q 1/6837

(57) ABSTRACT

The present application relates to an amino-modified chip, preparation method thereof and use thereof. The amino-modified chip comprises: a substrate and a high polymer, wherein the substrate is modified with epoxy groups; and the high polymer is attached to the substrate via the epoxy groups, at least one structural unit of the high polymer contains an amino, and the amino is a primary amino or a secondary amino.

### PRIORITY CLAIM

This application claims priority to International Application No. PCT/CN2021/111672, filed Aug. 10, 2021, which claims priority to Chinese Patent Application No. 202010807921.4, filed Aug. 12, 2020, the disclosures of which are incorporated herein by reference.

### REFERENCE TO SEQUENCE LISTING

This application contains a Sequence Listing submitted via EFS-Web. The entire contents of the sequence listing in ASCII text file entitled “GMB0005US_Sequence_Listing.txt” created on Feb. 8, 2023, and having a size of 2 kilobytes, is incorporated herein by reference.

### TECHNICAL FIELD

The present disclosure relates to the technical field of chips, and particularly, to an amino-modified chip, preparation method thereof and use thereof.

### BACKGROUND

With the gradual implementation of Human Genome Project and the rapid development of related disciplines in molecular biology, more and more genome sequences of animals, plants and microorganisms have been determined, and gene sequence data has increased rapidly at an unprecedented rate. The gene chip (also called DNA chip, biochip) technology, created with the need of such scientific development, is used for preparing and/or analyzing biomolecules which specifically refer to probe molecules (not limited to nucleic acid sequences) that are immobilized on a support and then hybridized with labeled sample molecules. The quantity and sequence information of the sample molecules are obtained by detecting the hybridization signal intensity of each probe molecule.

Chips are generally made by polymer-coated substrates. The analysis and/or preparation of molecules, such as some methods for nucleic acid sequencing, depend on the bonding of nucleic acid strands to the surface of the chip substrate, and the sequence of the bonded nucleic acid strand is then determined by a number of different methods well known in the art. Existing substrates are generally flow cells. Process for coating a flow cell may include transferring the polymerized mixture into channels on the flow cell and incubating for a fixed time. The process is simple and can produce reliable coatings that are always able to support all downstream chemical processing procedures including bridge amplification and sequencing.

However, existing chips are in need of further improvement, since they still have many defects or limitations limiting the applications thereof.

### SUMMARY

Based on this, it is necessary to provide an amino-modified chip. The amino-modified chip can carry a probe load of a higher density, and better meet the requirements of continuously developing biomolecule preparation and/or analysis with good stability.

Examples of the present disclosure provide an amino-modified chip, containing: a substrate modified with an epoxy group; and a polymer attached to the substrate via the epoxy group, where at least one structural unit of the polymer contains an amino group, and the amino group is a primary amino group and/or a secondary amino group.

In some embodiments, in the polymer, each of the structural unit contains an average of 0.05 to 10 identical or different amino groups.

In some embodiments, in the polymer, each of the structural unit contains an average of 1 to 5 identical or different amino groups.

In some embodiments, the polymer has a degree of polymerization in a range of 10 to 5000.

In some embodiments, in the polymer the structural unit contains at least one of

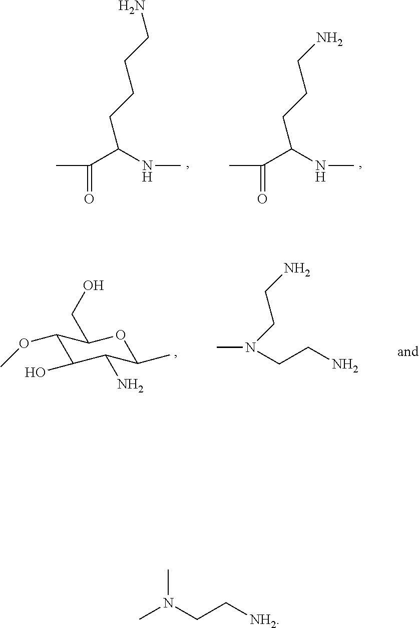

In some embodiments, the polymer is selected from at least one of polylysine, polyornithine, chitosan, a polyamidoamine dendrimer and polyethyleneimine.

In some embodiments, the chip further contains a probe attached to the polymer.

In some embodiments, the probe is bonded to the polymer via a linker compound, and the linker compound has a molecular structure containing a first linker group and a second linker group; the first linker group is bonded to the probe, and the second linker group is attached to the polymer through the amino group.

In some embodiments, the second linker group is selected from at least one of an —NHS group, an epoxy group and an isocyanate group.

In some embodiments, the linker compound is NHS-PEGₙ-DBCO or NHS-PEGₙ-N₃, where n is 3 to 2000, group —NHS in NHS-PEGₙ-DBCO or NHS-PEGₙ-N₃ is attached to the amino group via reaction.

In some embodiments, the probe is modified with group -DBCO or group —N₃; the bonding is a covalent bonding between group -DBCO and group —N₃.

Examples of the present disclosure further provide a method for preparing a chip, containing: (1) acquiring a substrate modified with an epoxy group; and (2) attaching a polymer to the substrate via reaction, where at least one structural unit of the polymer contains an amino group, and the amino group is a primary amino group or a secondary amino group.

In some embodiments, in the polymer, each of the structural unit contains an average of 0.05 to 10 identical or different amino groups.

In some embodiments, in the polymer, each of the structural unit contains an average of 1 to 5 identical or different amino groups.

In some embodiments, the polymer has a degree of polymerization in a range of 10 to 5000.

In some embodiments, in the polymer the structural unit contains at least one of

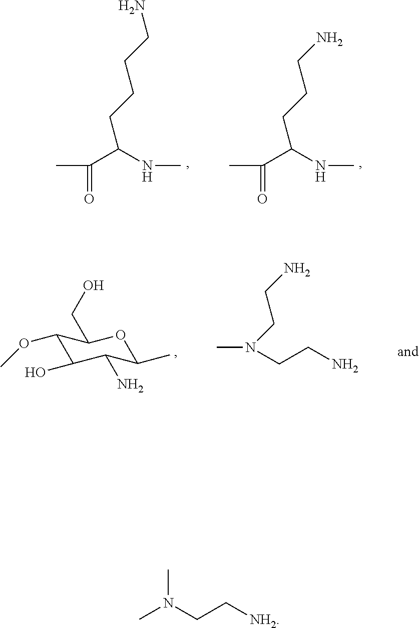

In some embodiments, the polymer is selected from at least one of polylysine, polyornithine, chitosan, a polyamidoamine dendrimer and polyethyleneimine.

In some embodiments, in step (2), the attaching via reaction is conducted by contacting the polymer with the substrate in an alkaline solution at pH 8.5 to 10.

In some embodiments, in step (2), the grating via reaction is conducted at a reaction temperature of 37 to 55° C. for 3 to 24 h.

In some embodiments, the method further contains step (3): attaching a probe to the polymer.

In some embodiments, the probe is attached to the polymer via a linker compound, and the linker compound has a molecular structure containing a first linker group and a second linker group; the first linker group is bonded to the probe, and the second linker group is attached to the polymer through the amino group.

In some embodiments, the second linker group is selected from at least one of an —NHS group, an epoxy group and an isocyanate group.

In some embodiments, the linker compound is NHS-PEGₙ-DBCO or NHS-PEGₙ-N₃, where n is 3 to 2000, group —NHS in NHS-PEGₙ-DBCO or NHS-PEGₙ-N₃ is attached to the amino group via reaction.

In some embodiments, the probe is modified with group -DBCO or group —N₃; the bonding is a covalent bonding between group -DBCO and group —N₃.

In some embodiments, the attaching via reaction is conducted by contacting the linker compound with the polymer in an alkaline solution at pH 7 to 9.

In some embodiments, the attaching via reaction is conducted at room temperature for 30 to 90 min.

In some embodiments, the covalent bonding is conducted by contacting the probe with the linker compound in an alkaline solution at pH 7 to 8.

In some embodiments, the alkaline solution for covalent bonding contains a surfactant selected from at least one of tetradecyltrimethylammonium bromide, cetyltrimethylammonium bromide and dodecyltrimethylammonium bromide.

Embodiments of the present disclosure further provides use of the chip as described above, or a chip prepared by the method as described above in preparation or analysis of a biomolecule.

The amino-modified chip disclosed in one embodiment of the present disclosure has a substrate modified with the specific active group, and the substrate is attached to a polymer containing a primary amino group and/or a secondary amino group via reaction. The polymer can form a modified surface with a higher density and a high reactivity for attaching a probe. The amino-modified chip can carry a probe load of a higher density, and better meet the requirements of continuously developing biomolecule preparation and/or analysis with good stability. The method for preparing a chip disclosed in another embodiment of the present disclosure can achieve the attaching of the probe without controlling reaction conditions strictly, making the preparing process for the chip simple, easy to control and favorable for popularization and application.

### BRIEF DESCRIPTION OF THE DRAWINGS

FIG. 1 is a schematic illustrating a preparation process of a chip in the examples, where X represents the polymer, and primer represents the primer/probe;

FIG. 2 is a graph illustrating probe density measurement results of chips prepared in Examples 1-5;

FIG. 3 is a graph illustrating DNA cluster detection results generated by the chip prepared in Example 1;

FIG. 4 is a graph illustrating DNA cluster detection results generated by the chip prepared in Example 2;

FIG. 5 is a graph illustrating DNA cluster detection results generated by the chip prepared in Example 3;

FIG. 6 is a graph illustrating DNA cluster detection results generated by the chip prepared in Example 4;

FIG. 7 is a graph illustrating DNA cluster detection results generated by the chip prepared in Example 5; and

FIG. 8 is a structural schematic of the DNA library used in Example 7.

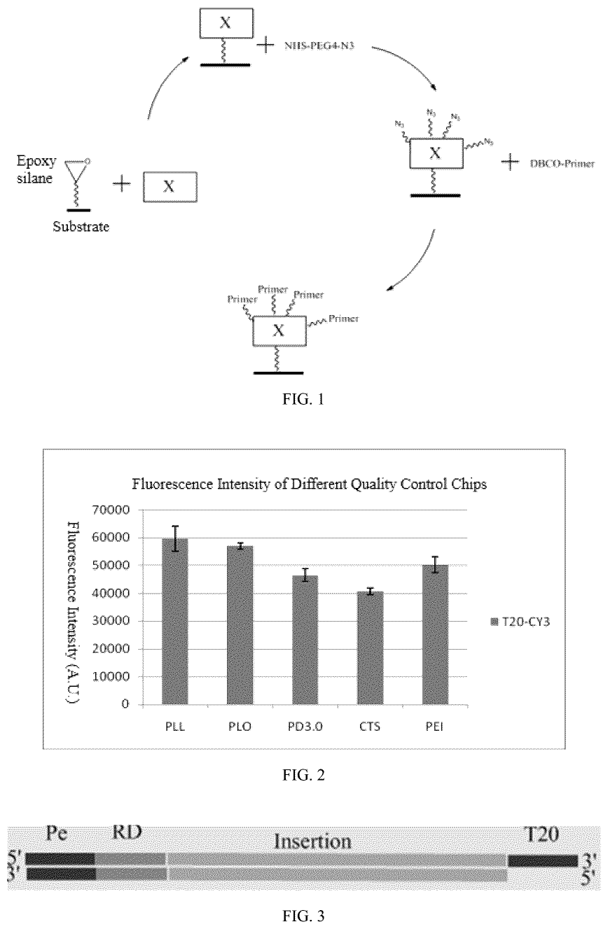

### DETAILED DESCRIPTION

The amino-modified chip of the present disclosure, the method for preparing the same and the use thereof are described in further details below with reference to specific examples. The present disclosure can be implemented in many different forms and is not limited to the embodiments described herein. In contrast, these embodiments are provided for a thorough and complete understanding of the present disclosure.

Unless defined otherwise, all technical and scientific terms used herein have the same meaning as commonly understood by those skilled in the art to which the present disclosure belongs. The terms used in the specification of the present disclosure herein are for the purpose of describing specific examples only and are not intended to limit the scope of the present disclosure. As used herein, the term “and/or” includes any and all combinations of one or more of the associated listed items.

The chip described herein may be a substrate to which the polymer is attached, or a substrate where a probe is further attached to the polymer. Materials for the substrate are not specified, and the substrate is formed of at least one of glass, silicon wafer, plastic, gel and nylon film.

The term “attached to” or “modified with” as used herein may refer to a direct attachment to or modification with an object, or may refer to a further attachment to or modification with the object via another transition group.

The amino group as used herein refers to a structural feature having a formula —N(X)₂, where each “X” is independently H, a substituted or unsubstituted alkyl, a substituted or unsubstituted cycloalkyl, a substituted or unsubstituted heterocyclyl, and the like. It will be appreciated that in the context of the present disclosure, at least one X is H. Non-limiting types of the amino group include —NH₂, —NH(alkyl), —NH(cycloalkyl), —NH(heterocyclyl) and —NH(aryl).

The term “alkyl” refers to a saturated hydrocarbon containing a primary (normal) carbon atom, a secondary carbon atom, a tertiary carbon atom, a quaternary carbon atom, or a combination thereof. Suitable examples include, but are not limited to: methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl(—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃ and octyl (—(CH₂)₇CH₃).

The term “cycloalkyl” refers to a non-aromatic hydrocarbon containing ring carbon atoms and may be a monocyclic, spirocycloalkyl or bridged cycloalkyl group. Suitable examples include, but are not limited to: cyclopropyl, cyclobutyl, cyclopentyl, cyclohexyl and cycloheptyl. In addition, “cycloalkyl” may also contain one or more double bonds, and representative examples of cycloalkyl groups containing double bonds include cyclopentenyl, cyclohexenyl, cyclohexadienyl and cyclobutadienyl.

The term “aryl” refers to an aromatic hydrocarbon group derived by removing one hydrogen atom from an aromatic cyclic compound, and may be a monocyclic aryl, fused cyclic aryl or polycyclic aryl. For polycyclic species, at least one of the rings is an aromatic ring system. Suitable examples include, but are not limited to: benzene, biphenyl, naphthalene, anthracene, phenanthrene, perylene, triphenylene, and derivatives thereof.

The term “heterocyclyl” refers to a cycloalkyl with at least one carbon atom replaced by a non-carbon atom, which may be an N atom, an O atom, an S atom, etc. The heterocyclyl may be a saturated ring or a partially unsaturated ring. Suitable examples include, but are not limited to: dihydropyridinyl, tetrahydropyridyl (piperidinyl), tetrahydrothienyl, thiooxidized tetrahydrothienyl, tetrahydrofuranyl, tetrahydroquinolinyl, tetrahydroisoquinolinyl and indolinyl.

The term “structural unit” refers to a unit structure formed by one of the monomers in the polymer. The polymer may be polymerized from one monomer or two or more different monomers, and the “structural units” may be identical or different.

The relationship between degree of polymerization and number of generation for polyamidoamine dendrimer: the degree of polymerization=2\hat{ } (number of generation+1)-1. For example, for PamamDendrimer generation 3.0, the degree of polymerization is 15.

Examples of the present disclosure provide an amino-modified chip, containing:
a substrate modified with an epoxy group; and a polymer attached to the substrate via the epoxy group, where at least one structural unit of the polymer contains an amino group, and the amino group is a primary amino group and/or a secondary amino group.

It will be appreciated that the chip can be used for genetic testing, or can be used for protein, polypeptide or other detections.

In one of the specific examples, in the polymer, each of the structural unit contains an average of 0.05 to 10 identical or different amino groups. It will be appreciated that the structural unit may or may not contain amino groups. The term “average” refers to a mean value obtained by dividing the total number of amino groups by the total number of the structural units. When the average number of amino groups in each structural unit is less than 1, the polymer is a copolymer obtained by polymerizing two or more monomers, at least one of which does not contain an amino group. For example, in the polymer each of the structural unit may contain an average of 0.5 amino groups, and the polymer may be composed of 19 structural units free of amino group and 1 structural unit containing amino groups (containing 10 amino groups). The number of amino groups can be controlled by controlling the combination of monomers in the polymerization.

Preferably, in the polymer, each of the structural unit contains an average of 1 to 5 amino groups. It is found that the number of amino groups in the polymer is associated with the reactivity of the amino groups, which directly influences the density/number of linkages, such as probes, between the amino groups and linkers connected to the polymer. In one example, the probes are linked to the surface of the chip through the linker compounds and the amino groups of the polymer. Controlling the average number of amino groups contained in each structural unit of the polymer at 1 to 5 can well regulate the linkage density/number of the probes, thus optimizing the regulation and control of the probe density. Meanwhile, the probes can be more easily attached to the polymer through the linker compounds with a higher probe density.

In one of the specific examples, the polymer has a degree of polymerization in a range of 10 to 5000. In another one of the specific examples, the polymer has a molecular weight of 30 kD to 300 kD. A higher molecular weight may result in a higher density of nucleic acid strand that can be attached. However, at the same time, it may increase the effectiveness of immobilization of the polymer on the surface of the substrate. Also, molecules with an excessively high molecular weight have undesirable effects in terms of solubility, reaction efficiency, and the like. Therefore, in the examples of the present disclosure, the molecular weight of the polymer is preferably 30 kD to 300 kD. For the polyamidoamine dendrimer, e.g., the PamamDendrimer, the number of generations may be 3 to 11.

Preferably, the polymer contains identical or different structural units, and may be commercially available or customized, as long as the requirements of the present application for the number of amino groups contained in the polymer and/or the degree of polymerization of the polymer are met.

In one of the specific examples, in the polymer the structural unit contains at least one of

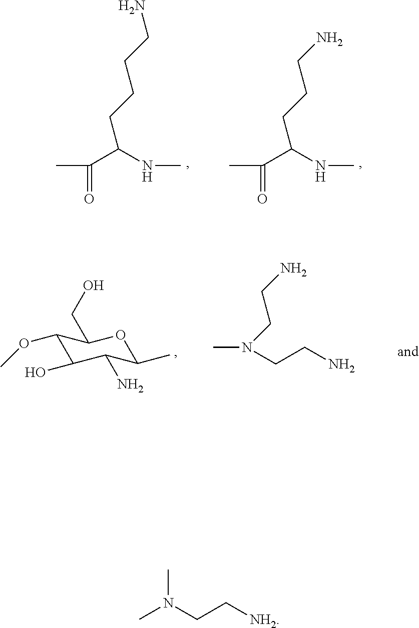

Specifically, in one example, the polymer contains identical structural units. For example, the polymer is a copolymer containing identical structural units of

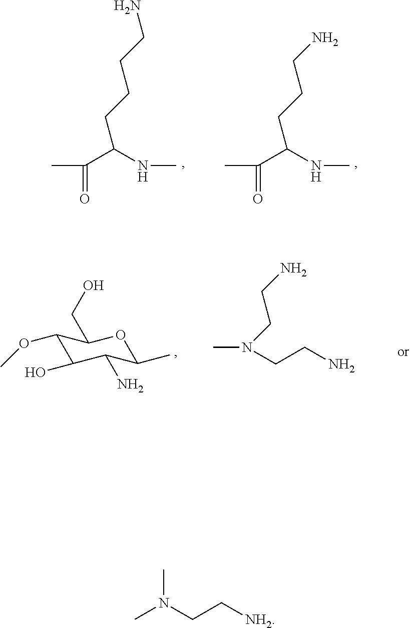

In another example, the polymer contains different structural units. For example, the polymer is a copolymer containing different structural units of

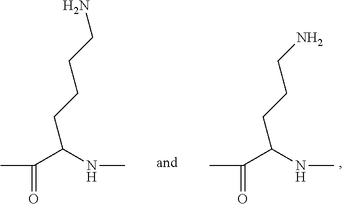

as disclosed in Kousaku Ohkawaa, “Biodegradation of Ornithine-Containing Polylysine Hydrogels”, Biomaterials 19(1998):1855-1860, including copolymers of ornithine and lysine polymerized at different ratios, or the polymer is a copolymer containing different structural units of

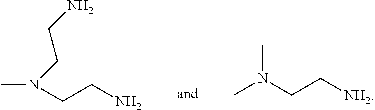

Preferably, the polymer is selected from at least one of polylysine, polyornithine, chitosan, a polyamidoamine dendrimer and polyethyleneimine.

In one of the specific examples, the chip further contains a probe attached onto the polymer. The probe binds to a target molecule and thus captures the target molecule. For different purposes, different probes may be selected. For example, oligonucleotide fragments, polypeptide sequences, probes containing oligonucleotide fragments, or probes containing polypeptide sequences can be selected. Meanwhile, the probes can selectively label fluorescent detection molecules, antigens, biotin, streptavidin, or other detection molecules.

In one of the specific examples, the probe may be a nucleic acid strand and/or a polypeptide.

In one of the specific examples, the probe is attached onto the polymer via a linker compound, and the linker compound has a molecular structure containing a first linker group and a second linker group; the first linker group at one end is bonded to the nucleic acid strand probe, and the second linker group at the other end is attached to the polymer through the amino group. Moreover, the second linker group is selected from at least one of an —NHS group, an epoxy group and an isocyanate group.

In one of the specific examples, the linker compound is made of NHS-PEGₙ-DBCO or NHS-PEGₙ-N₃, where n is 3 to 2000, group —NHS in NHS-PEGₙ-DBCO or NHS-PEGₙ-N₃ is attached to the amino group via reaction.

Preferably, the probe is modified with group -DBCO or group —N₃; the bonding is a covalent bonding between group -DBCO and group —N₃. NHS is an abbreviation for succinimidyl ester, PEG for polyethylene glycol, DBCO for diphenylcyclooctyne, N₃ for azide. The linkage between the nucleic acid strand and the polymer via the linker compound can, on one hand, allow NHS to be stably attached to the amino, and on the other hand, allow DBCO or N₃ to be subjected to a Click reaction with a nucleic acid strand modified with N₃ or DBCO under mild conditions of room temperature and no catalyst with high efficiency.

Examples of the present disclosure further provide a method for preparing a chip, containing: (1) acquiring a substrate modified with an epoxy group; and (2) attaching a polymer to the substrate via reaction, where at least one structural unit of the polymer contains an amino group, and the amino group is a primary amino group or a secondary amino group.

In the above method, further definitions of the polymer are the same as those for the chip described above and are not repeated hereinafter.

Specifically, step (1) is a substrate acquisition procedure. The acquisition can be a direct purchase, that is, acquiring a substrate modified with an epoxy group by direct purchase; or the acquisition can be self-making, where the process for self-making can be modifying a compound containing an epoxy group on the carrier by a solution reaction or plasma spray-coating.

The compound containing an epoxy group may be selected from an epoxy silane, such as 3-(2,3-glycidoxy)propyltrimethoxysilane. Additionally, in one of the specific examples, the material of the carrier is at least one of glass, silicon wafer, plastic, gel and nylon film.

In one of the specific examples, the reaction conditions for modifying the compound containing an epoxy group on the carrier by a solution reaction include: a reaction temperature at room temperature, and a reaction time of 1 to 8 h. Moreover, after the reaction, the mixture is dried at 80 to 150° C.

It will be appreciated that the carrier may require extensive washing and activation prior to the solution reaction modification. For example, the carrier surface is alternately washed with an alcohol solvent and water under ultrasonic conditions and activated with an alkaline solution. The alkaline solution activation can be conducted by processing, e.g., in a 0.1 M to 2 M aqueous NaOH solution for 1 to 20 min.

Specifically, step (2) is a polymer attachment procedure. The epoxy group on the substrate is modified, and in step (2), the amino group and the epoxy group are subjected to attaching via reaction.

Preferably, the attachment via reaction is conducted by contacting the polymer with the substrate in an alkaline solution at pH 8.5 to 10. The pH values include, but are not limited to: 8.5, 8.8, 9, 9.16, 9.2, 9.5 and 10.

Preferably, the alkaline solution at pH 8.5 to 10 is selected from at least one of a phosphate buffer, a carbonate buffer and a borate buffer. Furthermore, the concentration of the solute pair in the buffer at pH 8.5 to 10 is 10 to 300 mM. The concentrations of the solute pairs include, but are not limited to: 10 mM, 50 mM, 100 mM, 120 mM, 150 mM, 190 mM, 195 mM, 200 mM, 205 mM, 210 mM, 220 mM, 250 mM and 300 mM.

Preferably, a carbonate buffer at pH 9.16 is used as the reaction solvent, where the concentration of the solute pair is 200 mM.

Additionally, in step (2), the attachment via reaction is conducted at a reaction temperature of 37 to 55° C. for 3 to 24 h. The combination of reaction temperature and reaction time (temperature×time) includes, but is not limited to: 37° C.×14 h, 37° C.×16 h, 37° C.×18 h, 37° C.×20 h, 37° C.×24 h, 40° C.×12 h, 42° C.×18 h, 45° C.×10 h, 50° C.×8 h, 55° C.×5 h and 55° C.×3 h.

Preferably, the reaction temperature is 37° C. and the reaction time is 16 h.

Preferably, the method for preparing the chip described above further contains step (3): attaching a probe to the polymer. Step (3) is a probe attachment procedure.

In one of the specific examples, in step (3), the probe is attached to the polymer via a linker compound, and the linker compound has a molecular structure containing a first linker group and a second linker group; the first linker group is bonded to the probe, and the second linker group is attached to the polymer through the amino group. More specifically, the second linker group is selected from at least one of an —NHS group, an epoxy group and an isocyanate group.

Preferably, step (3) can be conducted in two procedures: (3-1) subjecting a compound providing the linker compound and an amino group of the polymer to a attachment reaction; and (3-2) bonding a product of the attachment reaction to a probe modified by an active group.

In one of the specific examples, the linker group is made of NHS-PEGₙ-DBCO or NHS-PEGₙ-N₃, where n is 3 to 2000, group —NHS in NHS-PEGₙ-DBCO or NHS-PEGₙ-N₃ is attached to the amino group via reaction. The linkage between the probe and the polymer via the linker compound can, on one hand, allow NHS to be stably attached to the amino, and on the other hand, allow DBCO or N₃ to be subjected to a click reaction with a probe modified with N₃ or DBCO under mild conditions of room temperature and no catalyst with high efficiency.

Preferably, the active group-modified probe is modified with group -DBCO or group —N₃; the bonding is a covalent bonding between group -DBCO and group —N₃. More specifically, the covalent bonding is a click reaction.

In one of the specific examples, the active group-modified probe is a nucleic acid strand modified by the active group at the 5′ end. It will be appreciated that if the linker compound is prepared from NHS-PEGₙ-DBCO, i.e., the group for bonding is -DBCO, the reactive group for 5′ modification is —N₃ functional group; if the linker compound is prepared from NHS-PEGₙ-N₃, i.e., the group for bonding is —N₃, the reactive group for 5′ modification is -DBCO functional group.

In one of the specific examples, in step (3-1), the concentration of the compound for providing the linker group in the reaction system is 50 μM to 5 mM. The concentrations include, but are not limited to: 50 μM, 100 μM, 500 μM, 800 μM, 1 mM, 1.2 mM, 1.5 mM, 2 mM and 5 mM.

In one of the specific examples, in step (3-1), the attachment via reaction is conducted by contacting the linker compound with the polymer in an alkaline solution at pH 7 to 9. Specifically, the pH values include, but are not limited to: 7, 7.3, 7.5, 7.7, 7.8, 7.9, 8, 8.1, 8.2, 8.3, 8.4 and 8.5. More specifically, the alkaline solution is selected from at least one of a phosphate buffer and a carbonate buffer.

In one of the specific examples, the concentration of the solute pair in the phosphate buffer or the carbonate buffer is 10 mM to 300 mM. The concentrations of the solute pairs include, but are not limited to: 10 mM, 50 mM, 80 mM, 90 mM, 95 mM, 98 mM, 100 mM, 102 mM, 105 mM, 110 mM, 150 mM, 200 mM, 210 mM, 220 mM, 250 mM and 300 mM.

Preferably, a phosphate buffer at pH 8 is used as the reaction solvent for the attachment via reaction, where the concentration of the solute pair is 100 mM. In another one of the specific examples, a carbonate buffer at pH 8.3 is used as the reaction solvent for the attachment via reaction, where the concentration of the solute pair is 100 mM.

In one of the specific examples, in step (3-1), the attachment via reaction is conducted at room temperature for 30 to 90 min. The reaction times include, but are not limited to: 30 min, 40 min, 50 min, 55 min, 58 min, 60 min, 62 min, 65 min, 70 min, 80 min and 90 min.

In one of the specific examples, in step (3-2), the covalent bonding is conducted by contacting the probe with the linker compound in an alkaline solution at pH 7 to 8. Specifically, the pH values include, but are not limited to: 7, 7.3, 7.5, 7.7, 7.8, 7.9 and 8. More specifically, the alkaline solution is a citrate buffer. More specifically, the covalent bonding is conducted at room temperature.

In one of the specific examples, the alkaline solution contains a surfactant selected from at least one of tetradecyltrimethylammonium bromide (TTAB), cetyltrimethylammonium bromide (CTAB) and dodecyltrimethylammonium bromide (DTAB). The surfactant is used to facilitate the sedimentation of the nucleic acid strands on the surface.

Preferably, the covalent bonding is conducted by contacting the probe with the linker compound in a citrate buffer containing 1 mM TTAB at room temperature.

Additionally, in various applications, excessive functional groups, such as —NH₂, -DBCO, —N₃, etc., may be optionally blocked before or after bonding to the probe.

Specifically, in one of the specific examples, the method further containing: conducting a blocking process using at least one of NHS-(PEG)ₙ, acetic anhydride, DBCO-benzoic acid and azidobenzoic acid, where n=3 to 2000. More specifically, the blocking of —NH₂ can be conducted using NHS-(PEG)ₙ(n can be 3 to 2000), acetic anhydride, etc.; specifically, for example, —NH₂ may be blocked using 1 μL of acetic anhydride+1.75 μL of N,N-diisopropylethylamine (DIPEA) in formamide. The blocking of —N₃ can be conducted using DBCO-benzoic acid. The blocking of -DBCO can be conducted using azidobenzoic acid.

Examples of the present disclosure further provides use of the chip as described above, or a chip prepared by the method as described above in preparation or analysis of a biomolecule. Specifically, the preparation or analysis of the biomolecule can be, for example, nucleic acid sequencing, library hybridization, generation of DNA clusters, and the like, and can also be, for example, protein detection, polypeptide detection, and the like.

In the following specific examples provided below, unless otherwise indicated, all experimental materials are either available from commercial suppliers (e.g., Aladdin™, Sigma™, Sangon™, etc.) or prepared in house or by a contractor in accordance with the structural formula and molecular weight information provided in the examples.

Polyamidoamine dendrimers (PamamDendrimer), or Pamam Dendrimer generation 3.0 (abbreviated as PD3.0), having a molecular weight of 6905.84 and the following molecular formula:

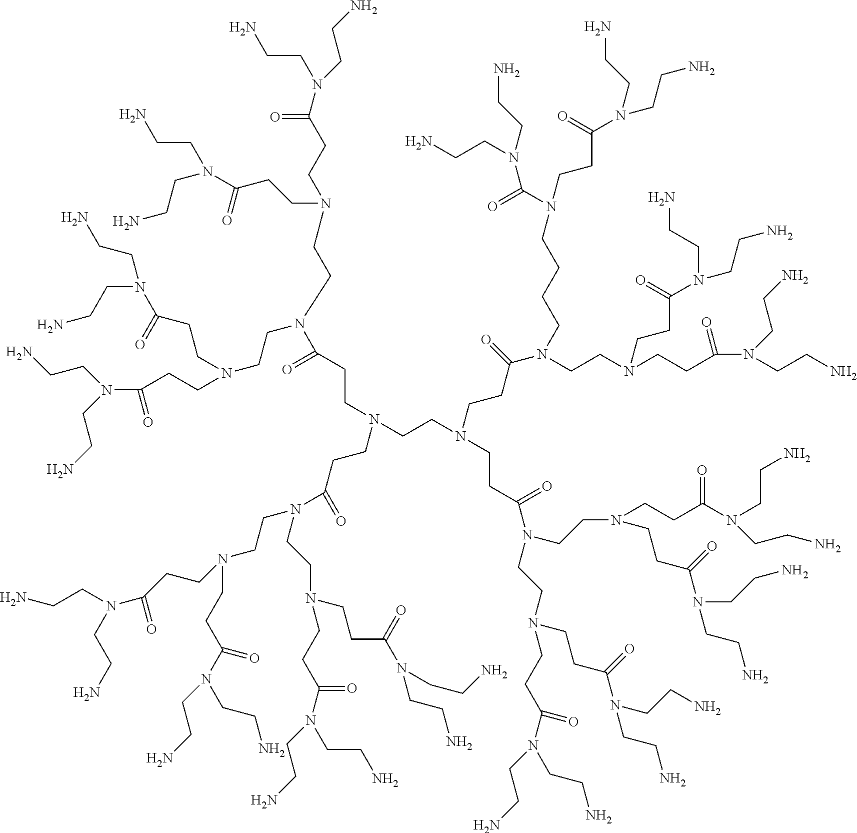 [SMILES: NCCN(CCN)C(=O)CCN(CCC(=O)N(CCN)CCN)CCN(CCCCN(CCC(=O)N(CCN)CCN)C(=O)N(CCN)CCN)C(=O)CCN(CCC(=O)N(CCN(CCC(=O)N(CCN)CCN)CCC(=O)N(CCN)CCN)CCN(CCC(=O)N(CCN)CCN)CCC(=O)N(CCN)CCN)CCN(CCC(=O)N(CCN(CCC(=O)N(CCN)CCN)CCC(=O)N(CCN)CCN)CCN(CCC(=O)N(CCN)CCN)CCC(=O)N(CCN)CCN)CCC(=O)N(CCN(CCC(=O)N(CCN)CCN)CCC(=O)N(CCN)CCN)CCN(CCC(=O)N(CCN)CCN)CCC(=O)N(CCN)CCN]

NHS-PEG₄-N₃ and NH—S-PEG₄-DBCO, having the following molecular formulas:

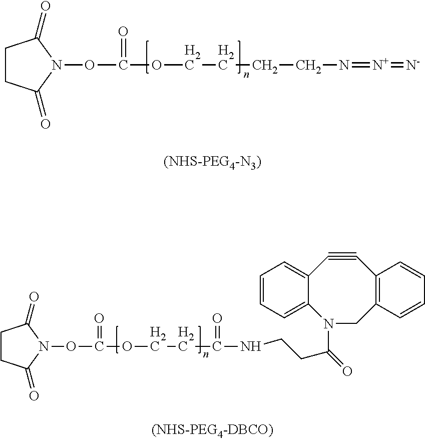

Anhydrous dimethyl sulfoxide (DMSO), purchased from Sigma™;

Polyethyleneimine(PEI),having a molecular weight of 60 kD and the following molecular formula:

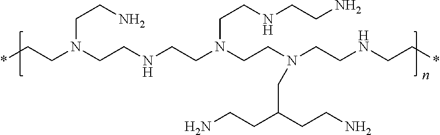

Polylysine (PLL), having a molecular weight of 150 to 300 kD and the following molecular formula:

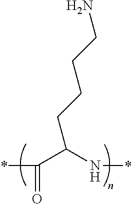

Polyornithine (PLO), having a molecular weight of 30 to 70 kD and the following molecular formula:

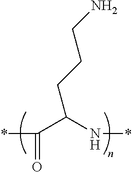

Chitosan, having a molecular weight of 100 kD and the following molecular formula:

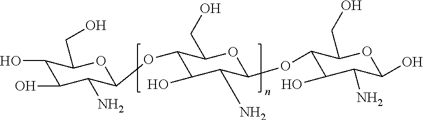

Sodium bicarbonate and sodium carbonate, purchased from Sigma™; Tetradecyltrimethylammonium bromide (TTAB), purchased from Sigma™; Disodium hydrogen phosphate and sodium dihydrogen phosphate, purchased from Sigma™; 20×SSC, purchased from Sangon™; Hepes, purchased from Sigma™; Tween 20, purchased from Sigma™; Sodium dodecyl sulfate (SDS), purchased from Aladdin™; SC: 0.2 M sodium carbonate, pH=9.16 (25° C.); PC: 0.1 M sodium phosphate, pH=8.0 (25° C.); Immobilization solution: 3×SSC+1 mM TTAB (tetradecyltrimethylammonium bromide); Washing solution 1:150 mM Hepes+0.1% Tween 20+0.1% SDS;

RI02: 3×SSC, pH=7.2 to 7.4

### Example 1

The specific procedures for coating the epoxy-modified substrate with PLO (polyomithine) and modifying the PLO-coated substrate with functional groups are as follows: 1. The 0.1% PLO solution was stored at room temperature for 15 min. 200 μL of SC buffer solution was added into 200 μL of the PLO solution. The mixture was well mixed by vortex, and centrifuged for 2 s. 2. The solution prepared in step 1 was introduced into lanes of an epoxy-modified substrate with 25 μL of solution into each lane. The lane ports were sealed (to prevent the solution from volatilizing). The substrate was incubated at 37° C. for 16 h. 3. After the reaction, when the substrate returned to room temperature, the substrate was washed with washing solution 1 and RI02, each 1 mL, on a fluid instrument. The parameters of the fluid instrument were: reagent amount=1000, speed=10, circle=1, delay=0, number=1. 4. The PLO-coated substrate was soaked in RI02 solution for storage at 4° C. for 1 week. 5. The PLO-coated substrate was modified with functional groups, which refer to groups that can participate in subsequent reactions, and may be —N₃, -DBCO and the like. In this example, the functional group was —N₃. The specific procedures of the functional group modification are as follows: 1) An NHS-PEG₄-N₃ solution with a final concentration of 100 mM was prepared with the solvent being anhydrous DMSO (anhydrous dimethyl sulfoxide); 2) A solution containing NHS-PEG₄-N₃ was introduced into the lanes of the PLO-coated substrate: On the fluid instrument, liquid in the lanes of the substrate was drained, the lanes were rinsed with 1 mL of PC buffer per lane, and the liquid in the lanes was drained after rinsing; 3) 396 μL of PC buffer and 4 μL of a 100 mM NHS-PEG₄-N₃ solution were mixed. The mixture was vortexed for mixing, and then centrifuged for 2 s; 4) The solution prepared in the step 3) was immediately introduced into lanes of the substrate with 25 μL per lane. The substrate was incubated for 1 h at room temperature; 5) After the reaction, the substrate was washed with washing solution 1, RI02 and ultrapure water, each 1 mL, on the fluid instrument; 6) The liquid in the lanes of the substrate was drained on the fluid instrument. 497.25 μL of formamide was added into a 1.5-mL centrifuge tube before 1.75 μL of DIPEA and 1 μL of acetic anhydride were sequentially added. The mixture was vortexed for mixing, centrifuged for 2 s, and immediately introduced into the lanes of the substrate with 25 μL per lane. The substrate was incubated for 15 min at room temperature. After the reaction, the substrate was washed with washing solution 1, RI02 and ultrapure water, each 1 mL, on the fluid instrument. 6. Probe immobilization: 1) On the fluid instrument, liquid in the lanes of the substrate was drained, the lanes were rinsed with 1 mL of immobilization solution per lane, and the liquid in the lanes was drained after rinsing; 2) 360 μL of the immobilization solution and 40 μL of a 100 μM A30 (SEQ ID No. 1) solution were mixed. The mixture was vortexed for mixing, centrifuged for 2 s, and immediately introduced into the lanes of the substrate with 25 μL per lane. The substrate was incubated for 16 h at 37° C.; 3) After the reaction, when the substrate returned to room temperature, the substrate was washed with washing solution 1 and RI02, each 1 mL, on a fluid instrument. The functional group-modified substrate can be soaked in RI02 solution for storage at 4° C. for 1 year.

### Example 2

The specific procedures for coating the epoxy-modified substrate with PLL (polylysine) and modifying the PLL-coated substrate with functional groups are as follows: 1. The 0.1% PLL solution was stored at room temperature for 15 min. 200 μL of SC buffer solution was added into 200 μL of the PLL solution. The mixture was well mixed by vortex, and centrifuged for 2 s. 2. The solution prepared in step 1 was introduced into lanes of an epoxy-modified substrate with 25 μL of solution into each lane. The lane ports were sealed (to prevent the solution from volatilizing). The substrate was incubated at 37° C. for 16 h. 3. After the reaction, when the substrate returned to room temperature, the substrate was washed with washing solution 1 and RI02, each 1 mL, on a fluid instrument. The parameters of the fluid instrument were: reagent amount=1000, speed=10, circle=1, delay=0, number=1. 4. The PLL-coated substrate was soaked in RI02 solution for storage at 4° C. for 1 week. 5. The PLL-coated substrate was modified with functional groups, which refer to groups that can participate in subsequent reactions, and may be —N₃, -DBCO and the like. In this example, the functional group was —N₃. The specific procedures of the functional group modification are as follows: 1) An NHS-PEG₄-N₃ solution with a final concentration of 100 mM was prepared with the solvent being anhydrous DMSO (anhydrous dimethyl sulfoxide); 2) A solution containing NHS-PEG₄-N₃ was introduced into the lanes of the PLL-coated substrate. On the fluid instrument, liquid in the lanes of the substrate was drained, the lanes were rinsed with 1 mL of PC buffer per lane, and the liquid in the lanes was drained after rinsing; 3) 396 μL of PC buffer and 4 μL of a 100 mM NHS-PEG₄-N₃ solution were mixed. The mixture was vortexed for mixing, and then centrifuged for 2 s; 4) The solution prepared in the step 3) was immediately introduced into lanes of the substrate with 25 μL per lane. The substrate was incubated for 1 h at room temperature; 5) After the reaction, the substrate was washed with washing solution 1, RI02 and ultrapure water, each 1 mL, on the fluid instrument; 6) The liquid in the lanes of the substrate was drained on the fluid instrument. 497.25 μL of formamide was added into a 1.5-mL centrifuge tube before 1.75 μL of DIPEA and 1 μL of acetic anhydride were sequentially added. The mixture was vortexed for mixing, centrifuged for 2 s, and immediately introduced into the lanes of the substrate with 25 μL per lane. The substrate was incubated for 15 min at room temperature. After the reaction, the substrate was washed with washing solution 1, RI02 and ultrapure water, each 1 mL, on the fluid instrument. 6. Probe immobilization: 1) On the fluid instrument, liquid in the lanes of the substrate was drained, the lanes were rinsed with 1 mL of immobilization solution per lane, and the liquid in the lanes was drained after rinsing; 2) 360 μL of the immobilization solution and 40 μL of a 100 μM A30 solution were mixed. The mixture was vortexed for mixing, centrifuged for 2 s, and immediately introduced into the lanes of the substrate with 25 μL per lane. The substrate was incubated for 16 h at 37° C.; 3) After the reaction, when the substrate returned to room temperature, the substrate was washed with washing solution 1 and RI02, each 1 mL, on a fluid instrument. The functional group-modified substrate can be soaked in RI02 solution for storage at 4° C. for 1 year.

### Example 3

The specific procedures for the coating epoxy-modified substrate with PamamDendrimer (polyamidoamine dendrimer) and modifying the PamamDendrimer-coated substrate with functional groups are as follows:
1. The 5% PamamDendrimer solution was stored at room temperature for 15 min. 380 μL of SC buffer solution was added into 200 μL of the PamamDendrimer solution. The mixture was well mixed by vortex, and centrifuged for 2 s.
2. The solution prepared in step 1 was introduced into lanes of an epoxy-modified substrate with 25 μL of solution into each lane. The lane ports were sealed (to prevent the solution from volatilizing). The substrate was incubated at 37° C. for 16 h.
3. After the reaction, when the substrate returned to room temperature, the substrate was washed with washing solution 1 and RI02, each 1 mL, on a fluid instrument. The parameters of the fluid instrument were: reagent amount=1000, speed=10, circle=1, delay=0, number=1.
4. The PamamDendrimer-coated substrate was soaked in RI02 solution for storage at 4° C. for 1 week.
5. The PamamDendrimer-coated substrate was modified with functional groups, which refer to groups that can participate in subsequent reactions, and may be —N₃, -DBCO and the like. In this example, the functional group was —N₃. The specific procedures of the functional group modification are as follows: 1) An NHS-PEG₄-N₃ solution with a final concentration of 100 mM was prepared with the solvent being anhydrous DMSO (anhydrous dimethyl sulfoxide); 2) A solution containing NHS-PEG₄-N₃ was introduced into the lanes of the PLO-coated substrate. On the fluid instrument, liquid in the lanes of the substrate was drained, the lanes were rinsed with 1 mL of PC buffer per lane, and the liquid in the lanes was drained after rinsing; 3) 396 μL of PC buffer and 4 μL of a 100 mM NHS-PEG₄-N₃ solution were mixed. The mixture was vortexed for mixing, and then centrifuged for 2 s; 4) The solution prepared in the step 3) was immediately introduced into lanes of the substrate with 25 μL per lane. The substrate was incubated for 1 h at room temperature; 5) After the reaction, the substrate was washed with washing solution 1, RI02 and ultrapure water, each 1 mL, on the fluid instrument.
6. Probe immobilization: 1) On the fluid instrument, liquid in the lanes of the substrate was drained, the lanes were rinsed with 1 mL of immobilization solution per lane, and the liquid in the lanes was drained after rinsing; 2) 360 μL of the immobilization solution and 40 μL of a 100 μM A30 solution were mixed. The mixture was vortexed for mixing, centrifuged for 2 s, and immediately introduced into the lanes of the substrate with 25 μL per lane. The substrate was incubated for 16 h at 37° C.; 3) After the reaction, when the substrate returned to room temperature, the substrate was washed with washing solution 1 and RI02, each 1 mL, on a fluid instrument; 4) The liquid in the lanes of the substrate was drained on the fluid instrument. 497.25 μL of formamide was added into a 1.5-mL centrifuge tube before 1.75 μL of DIPEA and 1 μL of acetic anhydride were sequentially added. The mixture was vortexed for mixing, centrifuged for 2 s, and immediately introduced into the lanes of the substrate with 25 μL per lane. The substrate was incubated for 15 min at room temperature. After the reaction, the substrate was washed with washing solution 1, RI02 and ultrapure water, each 1 mL, on the fluid instrument. The functional group-modified substrate can be soaked in RI02 solution for storage at 4° C. for 1 year.

### Example 4

The specific procedures for coating the epoxy-modified substrate with PEI (polyethyleneimine) and modifying the PEI-coated substrate with functional groups are as follows:
1. The 1% PEI solution was stored at room temperature for 15 min. 380 μL of SC buffer solution was added into 200 μL of the PEI solution. The mixture was well mixed by vortex, and centrifuged for 2 s.
2. The solution prepared in step 1 was introduced into lanes of an epoxy-modified substrate with 25 μL of solution into each lane. The lane ports were sealed (to prevent the solution from volatilizing). The substrate was incubated at 37° C. for 16 h.
3. After the reaction, when the substrate returned to room temperature, the substrate was washed with washing solution 1 and RI02, each 1 mL, on a fluid instrument. The parameters of the fluid instrument were: reagent amount=1000, speed=10, circle=1, delay=0, number=1.
4. The PEI-coated substrate was soaked in RI02 solution for storage at 4° C. for 1 week.
5. The PEI-coated substrate was modified with functional groups, which refer to groups that can participate in subsequent reactions, and may be —N₃, -DBCO and the like. In this example, the functional group was —N₃. The specific procedures of the functional group modification are as follows: 1) An NHS-PEG₄-N₃ solution with a final concentration of 100 mM was prepared with the solvent being anhydrous DMSO (anhydrous dimethyl sulfoxide); 2) A solution containing NHS-PEG₄-N₃ was introduced into the lanes of the PEI-coated substrate. On the fluid instrument, liquid in the lanes of the substrate was drained, the lanes were rinsed with 1 mL of PC buffer per lane, and the liquid in the lanes was drained after rinsing; 3) 396 μL of PC buffer and 4 μL of a 100 mM NHS-PEG₄-N₃ solution were mixed. The mixture was vortexed for mixing, and then centrifuged for 2 s; 4) The solution prepared in the step 3) was immediately introduced into lanes of the substrate with 25 μL per lane. The substrate was incubated for 1 h at room temperature; 5) After the reaction, the substrate was washed with washing solution 1, RI02 and ultrapure water, each 1 mL, on the fluid instrument. 6) The liquid in the lanes of the substrate was drained on the fluid instrument. 497.25 μL of formamide was added into a 1.5-mL centrifuge tube before 1.75 μL of DIPEA and 1 μL of acetic anhydride were sequentially added. The mixture was vortexed for mixing, centrifuged for 2 s, and immediately introduced into the lanes of the substrate with 25 μL per lane. The substrate was incubated for 15 min at room temperature. After the reaction, the substrate was washed with washing solution 1, RI02 and ultrapure water, each 1 mL, on the fluid instrument.
6. Probe immobilization: 1) On the fluid instrument, liquid in the lanes of the substrate was drained, the lanes were rinsed with 1 mL of immobilization solution per lane, and the liquid in the lanes was drained after rinsing; 2) 360 μL of the immobilization solution and 40 μL of a 100 μM A30 solution were mixed. The mixture was vortexed for mixing, centrifuged for 2 s, and immediately introduced into the lanes of the substrate with 25 μL per lane. The substrate was incubated for 16 h at 37° C.; 3) After the reaction, when the substrate returned to room temperature, the substrate was washed with washing solution 1 and RI02, each 1 mL, on a fluid instrument. The functional group-modified substrate can be soaked in RI02 solution for storage at 4° C. for 1 year.

### Example 5

The specific procedures for the coating epoxy-modified substrate with chitosan and modifying the chitosan-coated substrate with functional groups are as follows:
1. The 1% chitosan solution was stored at room temperature for 15 min. 380 μL of SC buffer solution was added into 20 μL of the chitosan solution. The mixture was well mixed by vortex, and centrifuged for 2 s.
2. The solution prepared in step 1 was introduced into lanes of an epoxy-modified substrate with 25 μL of solution into each lane. The lane ports were sealed (to prevent the solution from volatilizing). The substrate was incubated at 37° C. for 16 h.
3. After the reaction, when the substrate returned to room temperature, the substrate was washed with washing solution 1 and RI02, each 1 mL, on a fluid instrument. The parameters of the fluid instrument were: reagent amount=1000, speed=10, circle=1, delay=0, number=1.
4. The chitosan-coated substrate was soaked in RI02 solution for storage at 4° C. for 1 week.
5. The chitosan-coated substrate was modified with functional groups, which refer to groups that can participate in subsequent reactions, and may be —N₃, -DBCO and the like. In this example, the functional group was —N₃. The specific procedures of the functional group modification are as follows: 1) An NHS-PEG₄-N₃ solution with a final concentration of 100 mM was prepared with the solvent being anhydrous DMSO (anhydrous dimethyl sulfoxide); 2) A solution containing NHS-PEG₄-N₃ was introduced into the lanes of the PLO-coated substrate. On the fluid instrument, liquid in the lanes of the flowcell was drained, the lanes were rinsed with 1 mL of PC buffer per lane, and the liquid in the lanes was drained after rinsing; 3) 396 μL of PC buffer and 4 μL of a 100 mM NHS-PEG₄-N₃ solution were mixed. The mixture was vortexed for mixing, and then centrifuged for 2 s; 4) The solution prepared in the step 3) was immediately introduced into lanes of the flowcell with 25 μL per lane. The substrate was incubated for 1 h at room temperature; 5) After the reaction, the substrate was washed with washing solution 1, RI02 and ultrapure water, each 1 mL, on the fluid instrument. 6) The liquid in the lanes of the substrate was drained on the fluid instrument. 497.25 μL of formamide was added into a 1.5-mL centrifuge tube before 1.75 μL of DIPEA and 1 μL of acetic anhydride were sequentially added. The mixture was vortexed for mixing, centrifuged for 2 s, and immediately introduced into the lanes of the substrate with 25 μL per lane. The substrate was incubated for 15 min at room temperature. After the reaction, the substrate was washed with washing solution 1, RI02 and ultrapure water, each 1 mL, on the fluid instrument.
6. Probe immobilization: 1) On the fluid instrument, liquid in the lanes of the substrate was drained, the lanes were rinsed with 1 mL of immobilization solution per lane, and the liquid in the lanes was drained after rinsing; 2) 360 μL of the immobilization solution and 40 μL of a 100 μM A30 solution were mixed. The mixture was vortexed for mixing, centrifuged for 2 s, and immediately introduced into the lanes of the substrate with 25 μL per lane. The substrate was incubated for 16 h at 37° C.; 3) After the reaction, when the substrate returned to room temperature, the substrate was washed with washing solution 1 and RI02, each 1 mL, on a fluid instrument. The functional group-modified substrate can be soaked in RI02 solution for storage at 4° C. for 1 year.

### Example 6

In this example, the chips prepared in Examples 1 to 5 were subjected to a probe density detection. The specific detection procedures are as follows:
1. T20-CY3 hybridization: liquid in the lanes of the chips prepared in examples 1 to 5 was drained on the fluid instrument. 20 μL of hybridization solution A (3×SSC (pH 7.2 to 7.4) containing 3 μM T20-CY3) was introduced into each lane, and the chips were incubated at 55° C. for 15 min for hybridization. After cooling to room temperature, the chips were washed with 1 mL of RI02 on the fluid instrument. The Cy3 in the T20-CY3 was located at the 5′ end of the T20 sequence. Detection: Images were taken on a fluorescence detection system using a 20-fold objective lens, with a wavelength of 532 nm, a laser power of 35 mW, an exposure time of 60 ms, and were analyzed using ImageJ. The probe density detection results of the chips prepared in Examples 1 to 5 were shown in FIG. 2, where the ordinate represents the fluorescence intensity (A.U.) in unit dot/FOV, i.e., the mean fluorescence intensity of 1 observation region of 110×110 μm, and PLL, PLO, PD3.0, CTS and PEI on the abscissa respectively correspond to the chips modified with polymers PLL, PLO, PD3.0 (polyamidoamine dendrimer), CTS (chitosan) and PEI.

### Example 7

Library hybridization and detection were performed using the chips prepared in Examples 1 to 5. The specific procedures are as follows:

### 1. DNA Hybridization Library Preparation DNA Library:

DNA library of fragments with length of 150 to 300 bp and known sequences at both ends, the molecular structure of the library is shown in FIG. 3; Insertion: an inserted fragment derived from phi-X174 standard strain; T20, Pe and RD are respectively sequences set forth in SEQ ID Nos. 2 to 4. The DNA library was mixed with 52 μL of deionized water before 18 μL of freshly prepared 0.2 M NaOH solution was added. After well mixing, the mixture was let stand for denaturation at room temperature for 8 min, and then the reaction was stopped by adding 20 μL of 400 mM Tris-HCl buffer, pH 8.0 to obtain 100 μL of 100 μM denatured DNA library.

2. Hybridization of Denatured DNA Library with Chip Probes.

The denatured DNA library was diluted to 5 μM using a hybridization solution containing 3×SSC (20×SSC buffer diluted with RNase-free water), pH 7.3. The diluted DNA library was introduced into lanes of the chip and the chip was incubated for hybridization at 42° C. for 30 min. 160 to 260 μL of washing reagent (5×SSC, 0.05% Tween 20, pH 7.0) was introduced at a rate of 250 μL/min. The hybridization reaction was thus completed.

### 3. Initial Extension of the Template

1) 160 to 260 μL of extension buffer reagent (20 mM tris(hydroxymethyl)aminomethane (Tris), 10 mM ammonium sulfate, 2 mM magnesium sulfate, 1.5 M betaine, 1.3% dimethyl sulfoxide, 0.45 M N-methyl formamide, 1.5 M carboxamide, and 0.1% TritonX-100, pH 9.0) was introduced into the lanes of the chip at a rate of 500 μL/min;
2) 160 to 260 μL of extension reagent (extension buffer reagent, 3 μg/mL Bst DNA polymerase, 200 μM dNTPs) was introduced into the lanes of the chip at a rate of 500 μL/min, and the chip was incubated at 50 to 60° C. for 5 min to complete initial extension of the template.

### 4. DNA Cluster Generation

The amplification was carried out using the template walking technique disclosed in the article Isothermal amplification method for next-generation sequencing (Zhaochun Ma, et al, et.al, PNAS Aug. 27, 2013 110(35):14320-14323, https://doi.org/10.1073/pnas.1311334110).

### 5. DNA Cluster Detection

1) The thermal cycle temperature was set to 50 to 60° C.; 2) 160 to 260 μL of denaturing reagent formamide was introduced into lanes of the chip at a rate of 500 μL/min. The chip was incubated for 5 min to break the DNA double-helix structure; 3) 160 to 260 μL of quality control reagent (0.5 μM RD-Cy3, 3×SSC) was introduced into lanes of the chip at a rate of 500 μL/min, where Cy3 in RD-Cy3 was located at the 5′ end of RD sequence; 4) The thermal cycle temperature was set to 25° C. and the chip was incubated for 15 to 30 min for reaction; 5) 160 to 260 μL of washing reagent was introduced into lanes of the chip at a rate of 500 μL/min; 6) Images were taken on a fluorescence detection system using a 20-fold objective lens, with a wavelength of 532 nm, a laser power of 300 mW, and an exposure time of 20 ms.

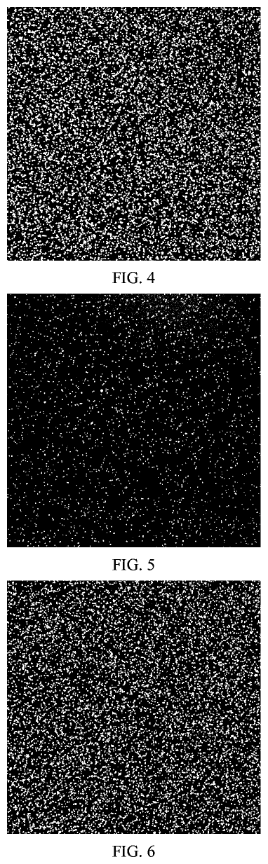

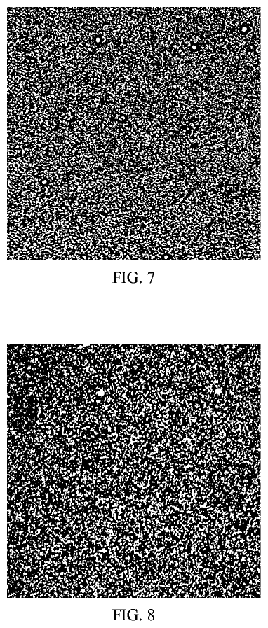

The results of DNA cluster detection are shown in FIGS. 3 to 7 and Table 1, and the results of DNA cluster detection corresponding to chips prepared in Examples 1 to 5 are shown in FIGS. 4 to 8, respectively.

TABLE 1

| | Density | Density | Average |
|---|---|---|---|
| | (cluster/ | (cluster/ | luminance per |
| Chip | μm²) | mm²) | cluster (cts) |
| Chip prepared in Example 1 | 0.07187 | 71870 | 1351 |
| Chip prepared in Example 2 | 0.01711 | 17110 | 743 |
| Chip prepared in Example 3 | 0.077666 | 77666 | 882 |
| Chip prepared in Example 4 | 0.143442 | 143442 | 3857 |
| Chip prepared in Example 5 | 0.106125329 | 106125 | 4336 |

As can be seen from the detection results, the chips prepare in Examples 1 to 5 were successfully used for hybridization of the library and DNA cluster generation.

Technical features in the above examples may be combined in any combinations. In order to make the description brief, all possible combinations of various technical features in the above examples are not described; however, it should be considered as being within the scope of this specification as long as there is no contradiction in the combinations of the technical features.

The above examples only illustrate one or more embodiments of the present disclosure for the purpose of specific and detailed description, but should not be construed as limiting the scope of the present disclosure. It should be noted that various changes and modifications can be made by those skilled in the art without departing from the spirit of the present disclosure, and these changes and modifications are all within the scope of the present disclosure. Therefore, the protection scope of the present disclosure should be determined with reference to the appended claim.

1. An amino-modified chip, comprising:
a substrate modified with an epoxy group; and
a polymer attached to the substrate via the epoxy group, wherein at least one structural unit of the polymer comprises an amino group, and the amino group is a primary amino group, and/or a secondary amino group.

2. The chip according to claim 1, wherein in the polymer, each of the structural unit comprises an average of 0.05 to 10 identical or different amino groups, optionally an average of 1 to 5 identical or different amino groups.

3. (canceled)

4. The chip according to claim 1, wherein the polymer has a degree of polymerization in a range of 10 to 5000.

5. The chip according to claim 4, wherein in the polymer the structural unit comprises at least one of

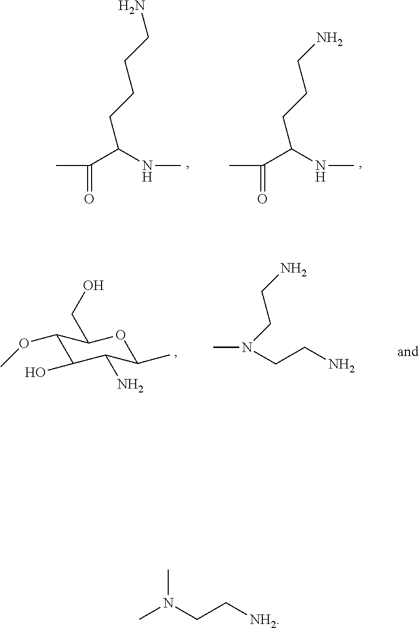

6. The chip according to claim 1, wherein the polymer is selected from at least one of a polylysine, a polyornithine, a chitosan, a polyamidoamine dendrimer, and polyethyleneimine.

7. The chip according to claim 1, further comprising a probe grafted attached to the polymer.

8. The chip according to claim 7, wherein the probe is attached to the polymer via a linker compound, and the linker group has a molecular structure comprising a first linker group and a second linker group; the first linker group is bonded to the probe, and the second linker group is attached to the polymer through the amino group.

9. The chip according to claim 8, wherein the second linker group is selected from at least one of an —NHS group, an epoxy group and an isocyanate group.

10. The chip according to claim 9, wherein the linker group is made of NHS-PEGₙ-DBCO or NHS-PEGₙ-N₃, wherein n is 3 to 2000, and group —NHS in NHS-PEGₙ-DBCO or NHS-PEGₙ-N₃ is attached to the amino group via reaction.

11. The chip according to claim 10, wherein the probe is modified with group -DBCO or group —N₃; the bonding is a covalent bonding between group -DBCO and group —N₃.

12. A method for preparing a chip, comprising:
attaching a polymer to a substrate modified with an epoxy group via reaction, wherein at least one structural unit of the polymer comprises an amino group, and the amino group is a primary amino group or a secondary amino group.

13. The method according to claim 12, wherein in the polymer,
(a) each of the structural unit comprises an average of 0.05 to 10 identical or different amino groups, optionally an average of 1 to 5 identical or different amino groups;
(b) the polymer has a degree of polymerization in a range of 10 to 5000;
(c) wherein in the polymer the structural unit comprises at least one of 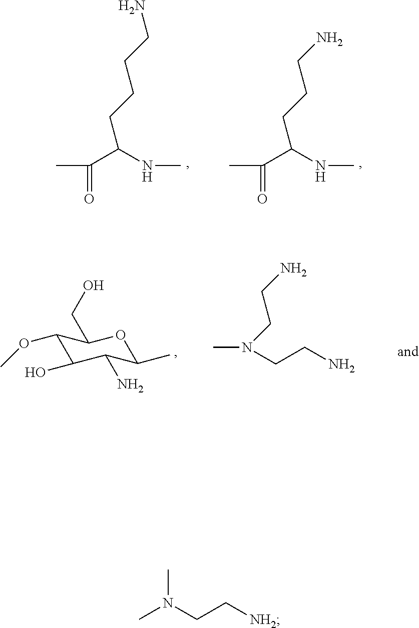
(d) wherein the polymer is selected from at least one of polylysine, polyornithine, chitosan, a polyamidoamine dendrimer and polyethyleneimine; or
(e) any combination of (a)-(d).

14-17. (canceled)

18. The method according to claim 12, wherein the attaching via reaction is conducted by contacting the polymer with the substrate in an alkaline solution at a pH in the range of 8.5 to 10.

19. The method according to claim 18, wherein the attaching via reaction is conducted at a reaction temperature in the range of 37 to 55° C. for a period of time in the range of 3 to 24 h.

20. The method according to claim 12, further comprising a step of: attaching a probe to the polymer.

21. The method according to claim 20, wherein the probe is attached to the polymer via a linker compound, and the linker compound has a molecular structure comprising a first linker group and a second linker group; the first linker group is bonded to the probe, and the second linker group is attached to the polymer through the amino group,
optionally wherein the second linker group is selected from at least one of an —NHS group, an epoxy group and an isocyanate group,
optionally wherein the linker group is made of NHS-PEGₙ-DBCO or NHS-PEGₙ-N₃, wherein n is 3 to 2000, and group —NHS in NHS-PEGₙ-DBCO or NHS-PEGₙ-N₃ is attached to the amino group via reaction,
optionally wherein the probe is modified with group -DBCO or group —N₃; the bonding is a covalent bonding between group -DBCO and group —N₃, or
optionally wherein the attaching via reaction is conducted by contacting the linker group with the polymer in an alkaline solution at pH 7 to 9.

22-24. (canceled)

25. The method according to claim 19, wherein the attaching via reaction is conducted by contacting the linker group with the polymer in an alkaline solution at a pH in the range of 7 to 9, optionally at a pH in the range of 7 to 8.

26. The method according to claim 21, wherein the attaching via reaction is conducted at room temperature for a period of time in the range of 30 to 90 min.

27. (canceled)

28. The method according to claim 19, wherein the alkaline solution for covalent bonding comprises a surfactant selected from at least one of tetradecyltrimethylammonium bromide, cetyltrimethylammonium bromide and dodecyltrimethylammonium bromide.

29. (canceled)

30. A method for analyzing a biomolecule comprising contacting a biomolecule with the chip of claim 1.


SEQUENCE LISTING (4 sequences)

SEQ ID NO: 1 - DNA, 30 bp, Artificial
  Adaptor
  misc_feature (1)..(30): Adaptor
aaaaaaaaaaaaaaaaaaaaaaaaaaaaaa

SEQ ID NO: 2 - DNA, 26 bp, Artificial
  Adaptor
  misc_feature (1)..(26): Adaptor
tttttttttttttttttttttttttt

SEQ ID NO: 3 - DNA, 27 bp, Artificial
  Adaptor
  misc_feature (1)..(27): Adaptor
caacaacaacaacaacaacaacaacaa

SEQ ID NO: 4 - DNA, 23 bp, Artificial
  Adaptor
  misc_feature (1)..(23): Adaptor
ctgccccgggttcctcattctat